(19) United States
(12) Patent Application Publication
(10) Pub. No.: US 2006/0239954 A1
(43) Pub. Date: Oct. 26, 2006

(54) Antimicrobial spray for use on pets

(76) Inventor: Karrie Ann Sancho, Yorktown, VA (US)

Correspondence Address: WILLIAMS MULLEN, FOUNTAIN PLAZA THREE, SUITE 200, 721 LAKEFRONT COMMONS, NEWPORT NEWS, VA, 23606, US

(21) Appl. No.: 11/409,826

(22) Filed: Apr. 24, 2006

Related U.S. Application Data
(60) Provisional application No. 60/673,957, filed on Apr. 22, 2005.

(51) Int. Cl. A61K 8/43

(52) U.S. Cl. 424/70.27; 424/70.28

(57) ABSTRACT

An antimicrobial liquid product is aqueous and/or alcoholic based for application to the feet of domesticated animals, particularly, the soles of the feet of domesticated animals, for the purpose of disinfecting or sanitizing the animal prior to the animal entering an area where pathogens are undesired. Examples of these areas may include residential homes, private businesses, restaurants, food preparation or processing facilities and motor vehicles. The product may also act as odor control and may also contain therapeutic properties. The antimicrobial efficacy can be tailored for residential and industrial applications.

### CROSS REFERENCE TO RELATED APPLICATION

The present application claims priority from U.S. Provisional Application Ser. No. 60/673,957, filed Apr. 22, 2005.

### BACKGROUND OF THE INVENTION

1. Field of the Invention

This invention relates to a formulation for disinfecting an animal's feet, and more particularly is concerned with a sprayable formulation for disinfecting a pet's feet.

2. Description of Related Art

Many devices and methods are known for sanitizing human hands. Humans are encouraged to use soap and water or antibacterial waterless lotions. These formulations act to kill germs on the user's hands. Devices for applying these products also exist.

U.S. Pat. No. 6,431,189 discloses a device for disinfecting hands. The user places their hands into a chamber through an opening. When a sensor detects hands, a solution is sprayed for a determined amount of time and then a fan provides drying air to dry the hands. U.S. Pat. No. 5,265,628 also discloses an automated cleansing chamber. This is a relatively complex machine in which two separate cylinders are provided, for a user's two hands. The cylinders are rotated and provide an array of nozzles to wash the user's hands. A program is provided comprising purge, wash, dry, rinse and self-clean cycles. Mechanically, the system is complex, and includes numerous moving parts, which would be subject to wear and deterioration.

While these are all options for humans, they are not suitable for animals for several reasons. In order to apply soap and water to an animal's feet, a human must come into contact with undesirable dirt and bacterial on the animal's feet as animals are unable to apply such solutions themselves. Animals are unlikely and unable to use automatic systems such as that described in the '189 patent discussed above. While humans are unlikely to lick their hands following treatment with a disinfectant, animals are. Thus any product used to disinfect the feet of animals must be safe for limited consumption.

An object of the present invention is to provide a disinfectant for animals.

An object of the present invention is to provide a disinfectant for sanitizing animals' feet.

An object of the present invention is to provide a disinfectant for sanitizing animals' feet which can be applied without touching the animal's feet.

An object of the present invention is to provide a disinfectant for sanitizing animals' feet which will not harm the animal if ingested by the animal licking its feet.

Finally, it is an object of the present invention to accomplish the foregoing objectives in a simple and cost effective manner.

### SUMMARY OF THE INVENTION

The present invention addresses these needs by providing a disinfectant solution for treating domesticated animals, which includes 2.0%-4.0% chlorhexidine gluconate and 0.1%-1.0% triclosan with the remainder of the solution made up of water, alcohol or a combination of water and alcohol. Preferably, the disinfectant solution as set forth in claim 1 further comprising a compound for healing skin damage, by preferably including 0.5-2.0% of allantoin in the solution. The disinfectant solution may alternatively include one or more of chlorhexidine gluconate, triclosan, quaternary ammonium compounds, povidone iodine, or quaternary ammonium compounds in combination with biguanides, all in amounts which are non-toxic to domestic animals with the remainder of the solution formed by a fluid selected from the group consisting of water and alcohol or a combination of water and alcohol. The solution is preferably applied to the animal by means of a spray bottle or a towlette.

### DETAILED DESCRIPTION OF THE PREFERRED EMBODIMENT

The following detailed description is of the best presently contemplated mode of carrying out the invention. This description is not to be taken in a limiting sense, but is made merely for the purpose of illustrating general principles of embodiments of the invention.

The present invention provides an antimicrobial liquid product which may be aqueous and/or alcoholic based for application to the feet of domesticated animals, particularly, the soles of the feet of domesticated animals, for the purpose of disinfecting or sanitizing the animal prior to the animal entering an area where such pathogens are undesired. Examples of these areas may include residential homes, private businesses, restaurants, food preparation or processing facilities and motor vehicles. The product may, but is not required, act as odor control. It may also contain therapeutic properties. The antimicrobial efficacy can be tailored for residential and industrial applications.

Generally, the disinfectant for domestic animals includes chlorhexidine gluconate, triclosan, quaternary ammonium compounds, povidone iodine, or quaternary ammonium compounds in combination with biguanides as active ingredients. A therapeutic substance such as allantoin is also preferably included.

The preferred formulation is an aqueous based solution containing the following components:
i. between 2.0% and 4.0% chlorhexidine gluconate (chlorhexidine digluconate C₂₂H₃₀CL₂N₁₀.2C₆H₁₂O₇) biguanide. Chlorhexidine gluconate has potent antimicrobial activity against most gram-positive and some gram-negative bacteria but not against spores. A 0.1% aqueous solution is bactericidal against Staphylococcus aureus, Escherichia coli, and Pseudomonas aeruginosa. However, it is relatively ineffective against other gram-negative organisms, spores, fungi, and most viruses. Its activity is unaffected or enhanced by alcohols. A 4% emulsion of chlorhexidine gluconate is used as a skin cleanser, a 0.5% (w/v) solution in 70% isopropanol as a general antiseptic, and a 0.5% solution in 70% isopropanol with emollients as a hand rinse. Chlorhexidine soaps have good residual activity. Chlorhexidine-alcohol mixtures are particularly effective in that they combine the antiseptic rapidity of alcohol with the persistence of chlorhexidine. Chlorhexidine has been incorporated into shampoos, ointments, skin and wound cleansers, teat dips, surgical scrubs, etc., because of its antiseptic properties and low potential for systemic or dermal toxicity. A 1% chlorhexidine acetate ointment is used as a topical antiseptic in treatment of external wounds in dogs, cats, and horses. Chlorhexidine is incompatible with anionic compounds, including soap;
ii. Between 0.1%-1.0% triclosan (polychlorophenoxyphenol—C₁₂H₄C₁₃O₂). Triclosan activity against gram-positive organisms (including Methicillin-resistant Staphylococcus aureus (MRSA)) is greater than against gram-negative bacilli, particularly P. aeruginosa. The agent possesses reasonable activity against mycobacteria and Candida spp; and
iii. Between 0.5-2.0% of allantoin (5-ureidohydantoin or glyoxyl-diureide or (2,5-dioxo-4-imidazolidinyl) urea—C₄H₆N₄O₃). Allantoin is a healing, moisturizing, soothing and anti-irritating, keratolytic and non-toxic agent useful in dermatological, cosmetic and veterinary preparation. Allantoin is a valuable cell-proliferating healing agent which stimulates healthy tissue formation. Allantoin has a pronounced keratolytic and skin softening action, acts as chemical debrider of necrotic and scaling tissue, cleaning the areas to which it is applied.

Additional embodiments of the disinfectant spray for domestic animals include quaternary ammonium compounds, povidone iodine, or quaternary ammonium compounds in combination with biguanides as active ingredients. These are acceptable as general use disinfectants to control negative bacteria and non-lipid-containing viruses. Exemplary solutions using these compounds include, in aqueous solution:
a. Between 0.1%-0.15% benzalkonium chloride (alkyl dimethyl benzyl ammonium chloride—[C₆H₅CH₂N(CH₃)₂R]Cl). Benzalkonium chloride is not compatible with anionic detergents; and is more effective against gram-positive bacteria than against gram-negative bacteria.
b. Between 0.05%-0.5% benzethonium chloride (methylbenzethonium chloride—C₂₇H₄₂ClNO₂). This compound is safe at concentrations of 0.5% and below in cosmetics applied to the skin.
c. Between 0.5%-10.0% povidone iodine (polyvinylpyrrolidone-iodine complex—(C₆—H₉—N—O)x.x-I₂). In mixtures of iodine with various surface-active agents, the iodophores show poor activity against bacterial spores but they are recommended for general use at concentrations of 75-150 ppm. They are effective against negative bacteria and viruses. The majority of iodophor preparations used for hand hygiene contain 7.5%-10% povidone-iodine. Formulations with lower concentrations also have good antimicrobial activity because dilution can increase free iodine concentrations. However, as the amount of free iodine increases, the degree of irritation also may increase; and
d. Between 50%-70% alcohols (C₃H₈O). Ethanol and isopropanol are the most common alcohol disinfectants used and they are most effective in 50%-70% aqueous solution, with isopropanol being slightly more potent.

In use, a handheld pump sprayer/mister is the preferred application method. Spray distance should minimize overspray and dispersal quantity should be of an amount that ensures desired coverage. Contact time of at least 15 seconds desired. The handheld pump is preferably manually operated to minimize frightening the animal as the solution is sprayed.

Alternate application may be by a handheld towlette. The solution may be provided on the towlette or an unprepared towlette may be sprayed with the solution for application on the animal. This method may be desired to thoroughly wipe a possibly contaminated area or with animals frightened by the spray.

Many improvements, modifications, and additions will be apparent to the skilled artisan without departing from the spirit and scope of the present invention as described herein and defined in the following claims.

What is claimed is:

1. A disinfectant solution for treating domesticated animals, comprising:
2.0%-4.0% chlorhexidine gluconate;
0.1%-1.0% triclosan; and
the remainder of the solution formed by a fluid selected from the group consisting of water, alcohol or a combination of water and alcohol.

2. The disinfectant solution as set forth in claim 1 further comprising a compound for healing skin damage.

3. The disinfectant solution as set forth in claim 1 further comprising 0.5-2.0% of allantoin.

4. A disinfectant solution for treating domesticated animals, comprising:
one or more of the compounds selected from the group consisting of chlorhexidine gluconate, triclosan, quaternary ammonium compounds, povidone iodine, or quaternary ammonium compounds in combination with biguanides, all in amounts which are non-toxic to domestic animals; and
the remainder of the solution formed by a fluid selected from the group consisting of water, alcohol or a combination of water and alcohol.

5. The disinfectant solution as set forth in claim 4 further comprising a compound for healing skin damage.

6. The disinfectant solution as set forth in claim 4 further comprising 0.5-2.0% of allantoin.

7. An apparatus for disinfecting the feet of a domesticated animal comprising:
a disinfectant solution comprising:
(a) 2.0%-4.0% chlorhexidine gluconate;
(b) 0.1%-1.0% triclosan; and
(c) the remainder of the solution formed by a fluid selected from the group consisting of water, alcohol or a combination of water and alcohol; and
an application means for applying the solution to the feet of the domesticated animal.

8. The apparatus as set forth in claim 7 wherein the means is selected from the group consisting of a spray bottle and a towlette.

9. The apparatus as set forth in claim 7 wherein the disinfectant solution further comprising a compound for healing skin damage.

10. The apparatus as set forth in claim 7 wherein the disinfectant solution further comprises 0.5-2.0% of allantoin.
